(19) United States
(12) Patent Application Publication
(10) Pub. No.: US 2018/0348324 A1
(43) Pub. Date: Dec. 6, 2018

(54) FAST METHOD FOR DETERMINING THE POSITION OF A FERROMAGNETIC PARTICLE OR A BUNCH OF FERROMAGNETIC PARTICLES USING MRI SYSTEMS

(71) Applicant: Bruker BioSpin MRI GmbH, Ettlingen (DE)

(72) Inventor: Arno NAUERTH, Erlenbach (DE)

(21) Appl. No.: 15/997,046

(22) Filed: Jun. 4, 2018

(30) Foreign Application Priority Data
Jun. 2, 2017 (DE) 10 2017 209 373.0

(51) Int. Cl. G01R 33/56; A61B 1/00; G01R 33/565; A61B 5/055; A61B 90/00; G01R 33/28

(52) U.S. Cl. CPC G01R 33/5601; A61B 1/00158; G01R 33/56572; A61B 2090/374; A61B 90/36; G01R 33/287; A61B 5/055

(57) ABSTRACT

A method for determining the position of at least one ferromagnetic particle in an object or a liquid matrix with an MRI system (50). An MRI measurement sequence is applied to a measurement volume (52), in which the particle is situated. At least two projections are recorded per spatial direction, wherein a read gradient is switched in each case for recording a one-dimensional projection in each case. A preselected parameter is modified for each of the two projections and the spatial position of the ferromagnetic particle is ascertained with the aid of the two projections. This facilitates a more reliable and precise determination of the position of a ferromagnetic particle or a bunch of ferromagnetic particles in a liquid matrix, wherein this position is determined in a very short time and requires the application of only a very low pulse in the process.

### CROSS REFERENCE TO RELATED APPLICATIONS

This application claims foreign priority under 35 U.S.C. § 119(a)-(d) to German Application No. 10 2017 209 373.0 filed on Jun. 2, 2017, the entire contents of which are hereby incorporated into the present application by reference.

### FIELD OF THE INVENTION

The invention relates to a method for determining the position of at least one ferromagnetic particle in an object using an MRI system, wherein an MRI measurement sequence is applied to a measurement volume, in which the particle is situated.

Such a method has been disclosed by U.S. Pat. No. 7,962,194 B2.

### BACKGROUND

Magnetic resonance imaging (=MRI) methods are used in diverse ways in order to obtain image information about structures. Here, it is also possible to obtain image information from the interior of a structure without damaging the structure. By way of example, internal views of the body parts of humans and animals are imaged using MRI methods in clinical applications.

Ferromagnetic particles within a magnetic field produce significant disturbance fields, the extent of which exceed a multiple of the dimensions of the particle. In conventional MRI measurement sequences, such disturbance fields appear as dark spots (“black holes”) at the core of the disturbance field and produce signal extinctions and image distortions to the outside. A first consequence of this is that the direct vicinity of the particle cannot be reproduced, or can only be reproduced inaccurately, by imaging MRI methods. Secondly, the precise determination of the location of ferromagnetic particles is found to be difficult and, as a rule, very time-consuming.

A general problem when using micro-machines, micro-robots or micro-components is that of deployment at a desired location (location of use). Dedicated drive systems, for instance walking legs, have been disclosed for micro-robots; however, such drive systems are expensive and difficult to construct. By contrast, in the case of ferromagnetic particles, there is the option of exerting a force thereon from the outside by way of a magnetic field gradient. As a result, it is possible to move the ferromagnetic particle contactlessly. In addition to movement, monitoring the current location of the ferromagnetic particle is necessary for the deployment at a desired location, in particular in order to correct the current location by further movements where necessary. However, if a magnetic particle is surrounded by a liquid matrix on the path to its location of use and if it needs to be moved through this liquid matrix, the position of the ferromagnetic particle in an image recording sequence often cannot be sensibly determined by way of the MRI system. The ferromagnetic particle often appears out of focus in the image and it is therefore not precisely localizable in the image recording or it may even have completely disappeared from the measurement volume.

U.S. Pat. No. 8,948,841 B2 describes a method for tracking a magnetic object with an MRI system, wherein the location of the magnetic object is calculated using projections of magnetic isosurfaces.

In order to obtain sufficient information for determining the position of the ferromagnetic particle, it is, fundamentally, required to perform a multiplicity of individual measurements that are worked through in sequence during a measurement sequence. After each of the individual measurements, a certain amount of time is allowed to elapse in order to allow the nuclear spins to relax. Forces act on the particle during the individual measurements and during the relaxation times, in particular as a result of gravity. These forces result from flows in the liquid matrix or else as a result of the spatially encoding gradient switching operations during the individual measurements. These forces result in shifting the particle during the measurement sequence. Shifts during the measurement sequence may corrupt the information for determining the position of the ferromagnetic particle. In the worst case scenario, the particle may even migrate out of the measurement volume.

In relation to the generic term “positive contrast MRI”, it is possible to find several examples of how accumulations of stem cells which are marked by ferromagnetic nanoparticles, for example, can be detected. Six such methods are described and compared by Evert-jan P. A. Vonken in an article in J Magn Reson Imaging. 2013 August; 38(2): 344-357 (“Direct in vitro comparison of six 3D positive contrast methods for susceptibility marker imaging”). Amongst others, the following methods are disclosed in this document:

WM (white marker): In this method, the shift of the MR signal in k-space is used to obtain a visible signal. This is achieved by changing the imaging gradients.

SGM (susceptibility gradient mapping) uses purely computational methods to shift the susceptibility change in voxels near the particle in k-space.

IRON (inversion recovery with on-resonant water suppression).

FSX (frequency selective excitation): This method makes use of off-resonance excitation pulses in the imaging part of the measurement sequence.

FLAPS (fast low angle positive contrast steady-state free precession): This method uses the phase rotation angle between successive RF excitations. There is a change in the angle with the change in the magnetic field if a susceptibility marker is present.

IDEAL (iterative decomposition of water and fat with echo asymmetry and least-squares estimation): Here, inter-echo phase shifts are used and combined with image post-processing.

A further method as described by Matthias Stuber in the article Magnetic Resonance in Medicine 58:1072-1077 (2007) (“Positive Contrast Visualization of Iron Oxide-Labeled Stem Cells using Inversion-Recovery With ON-Resonant Water Suppression (IRON)”), in which the off-resonance effects of spins near the ferromagnetic particle are exploited by virtue of saturating on-resonance spins and consequently only imaging spins in the vicinity of the ferromagnetic particles.

All of the aforementioned methods have in common that they require a relatively long recording time and additionally that they apply a high pulse power.

### SUMMARY

In contrast to this, it is an object of the present invention to facilitate a more reliable and precise determination of the position of a ferromagnetic particle or of a bunch of ferromagnetic particles in an object or a liquid matrix. It is a further object to determine this position in a very short time and by applying a pulse power that is as low as possible in the process.

These and other objects are achieved by virtue of, in a generic method of the type described above, at least two projections being recorded per spatial direction, wherein a read gradient is applied in each case for recording a one-dimensional projection in each case, wherein a preselected parameter is modified for each of the two projections and wherein the spatial position of the ferromagnetic particle is ascertained with the aid of the two projections.

In this method for determining the position of ferromagnetic particles that are only visible as artefacts in the MRI, the field disturbance in the surroundings of the particle, which spatially shifts the signals, is exploited. This shift can be made visible by different measurement methods and the accurate position of the particle or of a bunch of ferromagnetic particles can then be determined very quickly, exactly and without great technical outlay from the difference or the projections or from the superposition thereof.

The use of one-dimensional projections, optionally even without the use of spatial encoding gradients, makes the method according to the invention particularly simple and extraordinarily fast.

All that is required with this method is to change one sequence parameter for recording two projections per spatial direction. The field disturbance caused by the ferromagnetic particle brings about a faster decay of the FID, which, by implication, is effected by the modified parameter. Therefore, the disturbance can be localized with two projections when said parameter is varied.

In the simplest case, the measurement sequence only consists of radiating an excitation pulse and switching a read gradient with different readout parameters during the FID/Echo. In the case of the spin-echo method, a second pulse is required, which need not be 180° since a flip angle of 1° can also provide sufficient positional information. Further spatial encoding is not required for the one-dimensional projection.

The method according to the invention is typically configured as an operating method on an MRI system, wherein a control device is configured or programmed in such a way that at least two projections are recorded per spatial direction, wherein a read gradient is switched in each case for recording a one-dimensional projection, wherein a preselected parameter is modified for each of the two projections and wherein the spatial position of the ferromagnetic particle is ascertained with the aid of the two projections.

### Preferred Variants of the Invention

In a preferred class of embodiments of the method according to the invention, provision is made for the preselected, modified parameter to comprise a sign change in the read gradient. This can be used in the case of both spin-echo and gradient-echo measurements.

In advantageous variants of this class of embodiments, a spin echo method is used, in which the read gradients have different signs.

Developments of these method variants in which the RF pulse for the spin echo is designed for a flip angle <90° are particularly preferred. This method also works at a very small flip angle, for example of 1°. A 180° pulse is usually used for the spin echo, but the energy of the RF echo pulse radiated-in is lower at a lower flip angle; this is particularly gentle on the tissue.

In a further preferred class of embodiments of the method according to the invention, provision is made for the preselected, modified parameter to comprise a variation of the echo time. This applies both to the spin-echo time and to the gradient-echo time.

A third preferred class of embodiments of the method according to the invention is characterized in that the preselected, modified parameter comprises a variation in the amplitude of the read gradient while simultaneously maintaining the field of view. This results in a modified sweep width.

Embodiments of the method according to the invention in which two one-dimensional projection images are recorded in each case in three different, preferably orthogonal, spatial directions and the spatial position of the ferromagnetic particle in the three-dimensional space is determined therefrom are also advantageous. This is particularly expedient if there is no a priori knowledge as to the location of the particle. Then, the entire surroundings will be “combed through”, as it were.

In further embodiments, it may be advantageous if the position of the ferromagnetic particle is determined by forming the difference of the recorded one-dimensional projection images or by overlaying the recorded one-dimensional projection images or by using pattern recognition when comparing the recorded one-dimensional projection images to one another.

Embodiments of the method according to the invention that are distinguished in that the ferromagnetic particle is marked with another NMR-active nucleus, e.g., by coating, are also very particularly preferred. A substantial advantage here is that there are substantially more uniform surroundings around the particle when recording a projection with a different frequency that is not identical to the proton frequency. By way of example, if the uncoated particle rests against the intestinal wall of a biological examination object, it is possible that there is a gas bubble, which contains no 1H nucleus, in the surroundings. A very inaccurate projection then results therefrom. By way of example, the same also applies to different neighbouring tissues (bones, fatty tissue, liquid, etc.). By way of example, if the particle is coated by Teflon, i.e. by polytetrafluoroethylene (PTFE), the surroundings of the particle are always provided homogeneously with 19F nuclei.

In advantageous developments of these embodiments, spin-active nuclei can be used as marker or coating material, preferably those that can be found as heteroatoms in polymers, such as F in Teflon, Cl in PVC, N in polyurethanes, in particular 6Li, 10B, 14N, 15N, 17O, 19F, 23Na, 29Si, 31P, 35Cl, 113Cd, 195Pt. Advantageously, it is possible to use materials that can serve for coating purposes, preferably inert materials such as, for instance, PVC, PTFE, silicone (polysiloxanes), polyphosphazene (31P), polyurethanes (15N), optionally also polyether or polyester (17O). Although it would be possible to choose Pt, this is relatively expensive. Cd could likewise be used in theory, but it is poisonous.

The MRI signals from the measurement volume can be produced through a spin-echo method (e.g. MSME) or through a gradient-echo method, in particular using the FLASH method, or the ZTE method or through the UTE method or through the SPI method.

### MRI System According to the Invention

The scope of the present invention furthermore includes an MRI system, comprising a magnet for producing a homogeneous magnetic field B₀ in a measurement volume, a gradient coil system for producing magnetic field gradients—in particular spatially encoding magnetic field gradients as well—in the measurement volume, and a radiofrequency excitation and read coil system for radiating radiofrequency pulses into the measurement volume and for reading the measurement volume, which is characterized in that the MRI system is configured to determine the position of a ferromagnetic particle with an above-described method according to the invention. The MRI system or the control device has appropriate programming and facilitates, in a simple fashion, fast and rather accurate monitoring of the position of ferromagnetic particles.

Further advantages of the invention emerge from the description and the drawing. The features mentioned above and the features yet to be explained below may also, according to the invention, find use on their own in each case or together in any combination. The shown and described embodiments should not be understood to be a complete list but, instead, have an exemplary character for explaining the invention.

### BRIEF DESCRIPTION OF THE DRAWINGS

The invention is illustrated in the drawing and will be explained in more detail on the basis of exemplary embodiments. In the figures:

FIG. 1 shows field disturbances, imaged by different measurement methods;

FIG. 2 shows field disturbance, recorded using MSME;

FIG. 3 shows field disturbances in the XZ-plane and the associated projection, recorded using MSME with positive and negative gradient switching;

FIG. 4A shows field disturbances caused by magnetic particles in the case of different gradient-echo times (1 ms and 2 ms) and the respective 1D projections;

FIG. 4B shows a centre of mass of the difference projection;

FIG. 5 shows the influence of the sweep width on the field disturbances;

FIG. 6 shows the localization of a magnetic particle by exploiting an asymmetric artefact with the aid of opposing read gradients; and

FIG. 7 shows a schematic illustration of an MRI system for carrying out the method according to the invention.

### DETAILED DESCRIPTION

The present invention relates to a method for an improved determination of the position of a ferromagnetic particle in an object, preferably in a fluid matrix, using a magnetic resonance imaging (=MRI) system.

The above-noted objects according to one aspect of the invention are addressed by virtue of one-dimensional projections being produced in a suitable manner, said one-dimensional projections allowing the position of a ferromagnetic particle or of a bunch of ferromagnetic particles to be determined.

Here, one solution according to the invention is based on the discovery that the magnetic field is disturbed in the direct vicinity of the ferromagnetic particle in such a way that the nuclear spins situated there are modified in terms of their magnetic encoding by the gradients. Furthermore, the field disturbance of the particle brings about stronger de-phasing of the spins in the direct vicinity of the particle and therefore imaging with a lower intensity in the projection. As a consequence of this local field disturbance, the signals that are produced by the nuclear spins situated there are spatially out of place. The shifts are made visible or measurable in a projection.

Compared to known methods, the method according to a further aspect of the invention is distinguished in that MRI signals are recorded from the measurement volume, said MRI signals having artificial spatial shifts on account of magnetic field disturbances in the surroundings of the ferromagnetic particle caused by the presence of the ferromagnetic particle in the NMR magnetic field in the measurement volume, and in that, subsequently, the spatial position of the ferromagnetic particle is ascertained with the aid of these measured spatial shifts in the MRI signals.

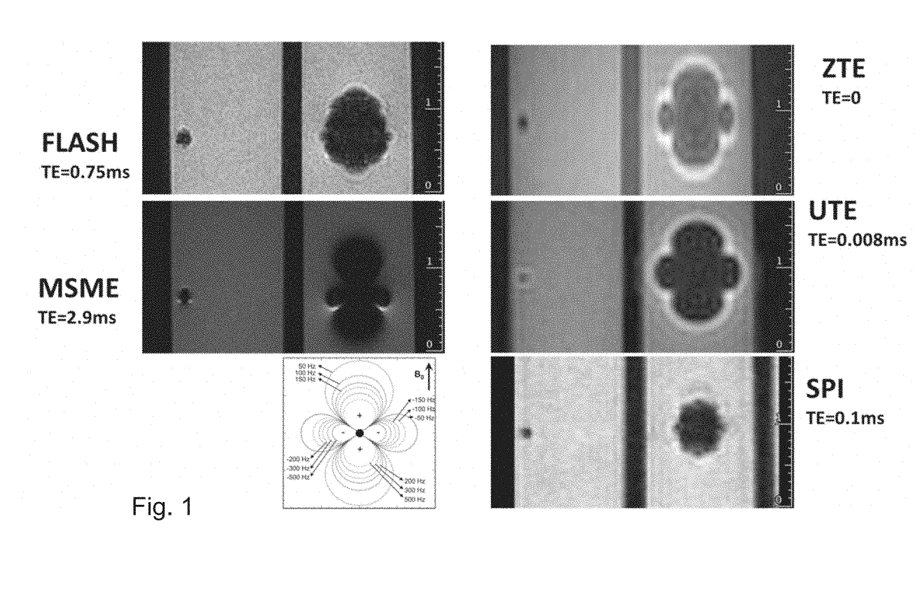

FIG. 1 shows a plurality of MRI recordings, wherein two ferromagnetic particles of different sizes are presented in a gel matrix. A sample vessel with a particle with a diameter of 0.35 mm is found on the left-hand side in each case; the right-hand side of the recording is a sample vessel with a particle with a diameter of 1 mm The manner in which a field disturbance caused by the magnetic particles affects different measurement methods in the MRI is illustrated. It is clearly visible in each case that the exact position of the particle is not exactly determinable since the surroundings of the particle in the recorded image appear as an out of focus cloud on account of the strong field disturbances caused by the particle.

The stated problem of a fast and accurate determination of the position of the particle to be examined is now solved to the effect of producing a one-dimensional projection in each spatial direction using one of the different measurement methods, therewith allowing to localize the particle precisely. This method even works if the particle itself does not have a perfectly symmetrical form (e.g. round or elliptic) since the spins in the entire surroundings of the particle appear symmetrically displaced in the projection.

The following examples, which were created with the aid of both spin-echo and gradient-echo sequences illustrate the principles of the invention:

### Measurement Sequences According to the Invention

### Example 1: Spin-Echo (MSME):

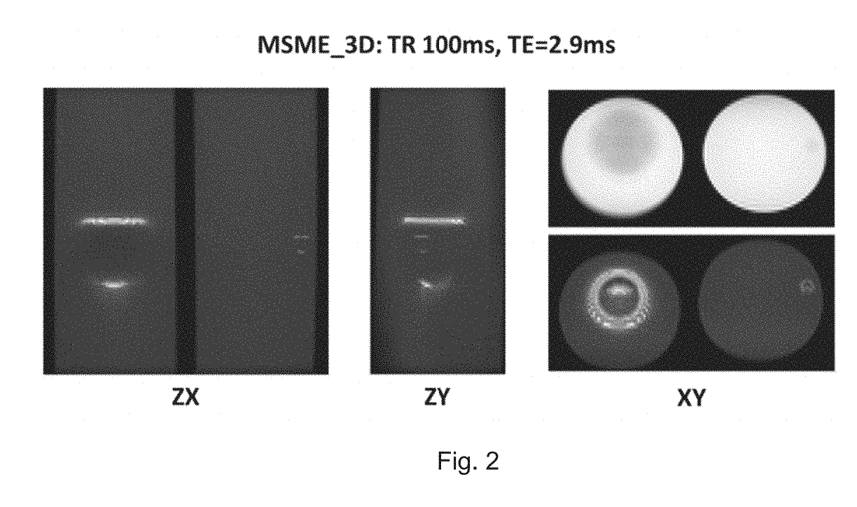

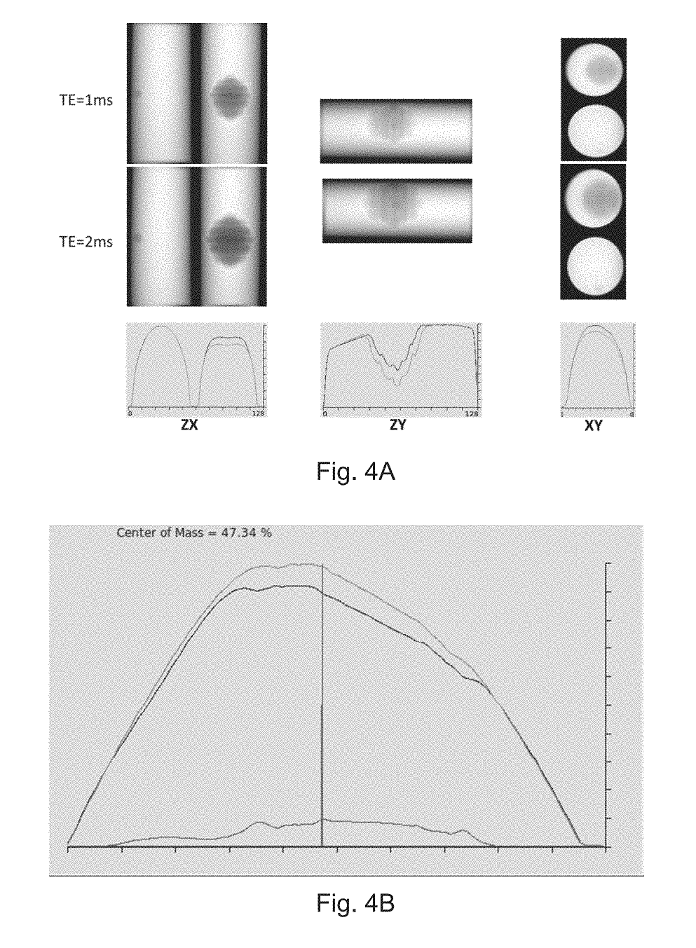

The effect that the magnetic particle has on MR imaging using a spin-echo method (MSME) is visible in FIG. 2. Transversely to the B₀-field, i.e., in the ZX-plane and in the ZY-plane, it is possible in each case to identify a bright stripe that lies at a slight distance from the centre of the artefact. In the XY-plane (as seen in the direction of the B₀-field), it is possible to identify a bright ring that extends symmetrically around the centre of the particle. The recording was made with an echo time TE=2.9 ms. The diameter of the ring artefact in the XY-plane changes with the echo time. Thus, it is possible, for example, also to make two measurements with different TE and, on the basis thereof, use the projections for calculating the exact position of the particle (see FIG. 4).

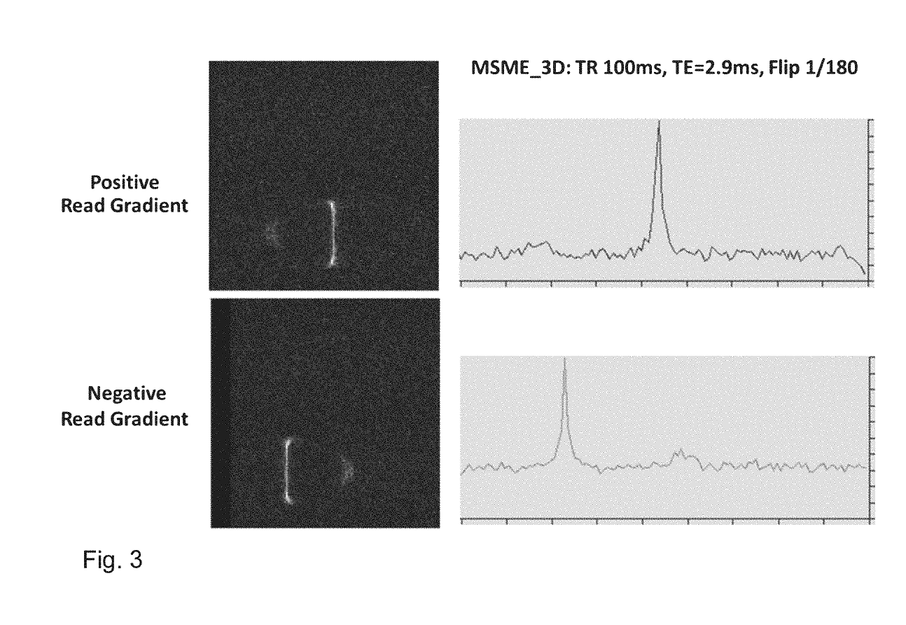

The determination is not clear in the planes perpendicular to the B₀-field since the projection is asymmetric. In this case, use is made of additional switching of a read gradient, which is switched once with a positive sign and once with a negative sign. It is clear from FIG. 3 that two very sharp projections are obtained, said projections being equidistant from a centre at which the magnetic particle is situated.

The spin-echo method is disadvantageous in that a relatively high energy is radiated-in with the RF pulse. However, for recording the projection as shown in the exemplary recordings in FIG. 3, no full 180° pulse is required; a projection is also measurable at flip angles of less than 90°, although this depends on the size of the particle. The present recordings were created by a 1° flip angle, with it being difficult to make out the smaller particle with the 0.35 mm diameter. The visible recordings relate to the particle with the larger diameter of 1 mm.

### Example 2: Change in the Echo Time (Gradient Echo):

It is clear from FIG. 4A that the artefact increases in width with the gradient echo time, to be precise in each of the three spatial planes. The echo time causes a symmetric propagation of the disturbance. This effect can be used to draw conclusions about the accurate position of the particle.

By way of example, the position of a magnetic particle is ascertained by virtue of determining the centre of mass of the difference of the two projections: FIG. 4B shows a projection with TE=1 ms (uppermost curve), a projection with TE=2 ms (central curve) and the difference function (lowest curve). Here, the ascertained centre of mass was at 47.34% of the projection direction. Consequently, in the case of a projection of 10 cm, the magnetic particle would be situated 4.734 cm from the left-hand edge or 2.66 cm to the left of centre.

### Example 3: Change in the Sweep Width (Gradient Echo):

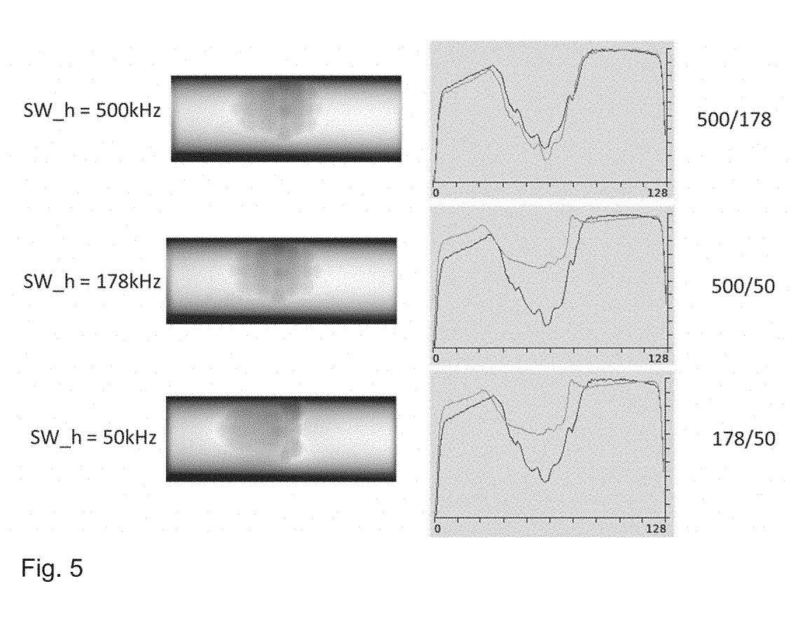

The change in the sweep width (bandwidth of the digitization) is accompanied by a change in the strength of the read gradient such that the imaging range (field-of-view) remains unchanged. FIG. 5 shows that the disturbance by the magnetic particle in the image becomes asymmetrical in the case of different digitization times. The disturbance is deformed and also shifted away from the centre point. In order to evaluate this measurement artefact in respect of determining the position, it is possible to use pattern recognition in which a plurality of projections with different bandwidths are used. Here, single calibration measurements, for example, are carried out, in which the characteristic disturbances, the difference function thereof and the associated deviation of the particle position from the centre of mass of the difference function are ascertained.

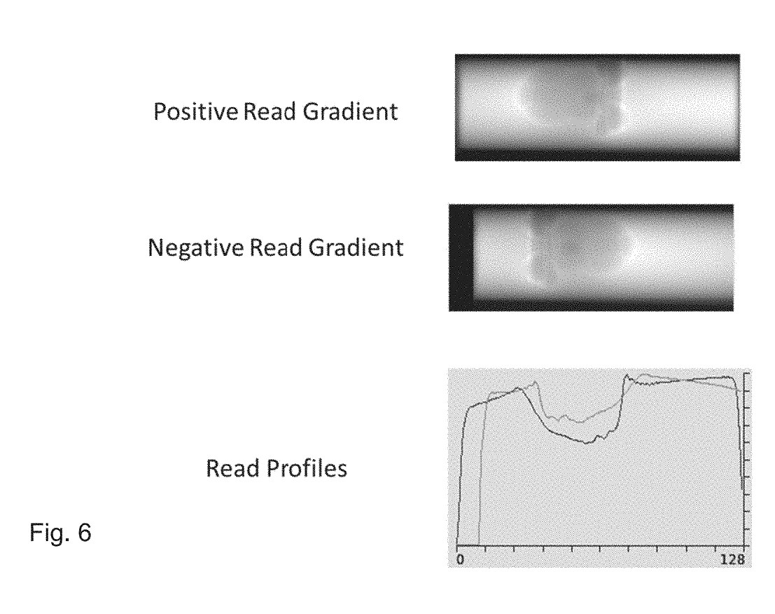

The asymmetry, which is identifiable at 50 kHz in FIG. 5, for example, can also be used, once again, to invert the read gradient, similar to Example 1 above, such that a unique position can be localized from the respectively opposing projection of positive and negative gradient switching, as can be seen in FIG. 6, which shows the localization of a magnetic particle using an asymmetric artefact with the aid of inverted read gradients.

The exemplary recordings of magnetic particles shown in the figures of the drawing were created in homogeneous surroundings, more precisely in a gel. Consequently, the 1H spins in the surroundings of the magnetic particle are under the same influence of the neighbouring spins. There is a different reaction in the case of imaging in a non-homogeneous object, such as in the bodies of animals and humans, for example. The spin-spin and the spin-lattice relaxation times change in different tissues and the 1H nucleus densities are very variable. An extreme example would be a signal-free space in a gas bubble. If the particle were in this position, it could only be localized with difficulty. The inhomogeneity of the tissue also makes the localization with the aforementioned method more difficult. In a preferred embodiment, the magnetic particle can be marked by another NMR-active nucleus. In principle, all NMR-active nuclei can use such a tracer material. These would then be read out by the corresponding frequency, and so this always yields the same disturbance images, independently of the neighbouring tissue structures.

By way of example, all spin-active nuclei can be used here as tracer material (6Li, 10B, 14N, 15N, 17O, 19F, 23Na, 29Si, 31P, 35Cl, 113Cd, 129Xe, 195Pt); however, use is preferably made of those that can be found as heteroatoms in polymers, such as F in Teflon, Cl in PVC, N in polyurethanes, etc.

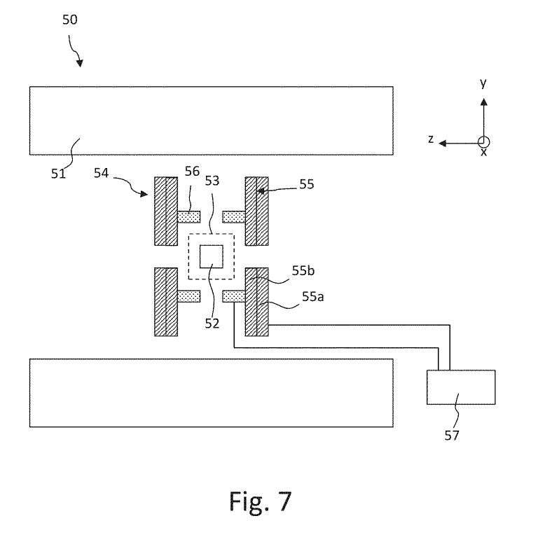

Finally, FIG. 7 shows an embodiment of an MRI system 50 according to the invention, in particular for implementing a determination of position and/or a change in position of a ferromagnetic particle (object) within the scope of the present invention.

The MRI system 50 has a magnet (main magnet coil system, typically superconducting) 51, with which a strong, homogeneous magnetic field B₀ can be produced in a horizontal z-direction in a measurement volume 52, typically with a strength of 1T or more. Using a radiofrequency excitation and read coil system 53, it is possible to radiate RF pulses into the measurement volume 52 and an RF signal of the measurement volume can be read out. Furthermore, provision is made of a gradient coil system 54, with which magnetic field gradients can be produced in the measurement volume 52.

Here, the gradient coil system 54 comprises a first coil subsystem 55, with which magnetic field gradients can be produced in a vertical y-direction; i.e., the magnetic field strength changes along the y-direction. Furthermore, provision is made of a second coil subsystem 56, with which magnetic field gradients can be produced in the horizontal z-direction; i.e., the magnetic field strength changes along the z-direction.

The gradient coil system 54 is controlled by way of an electronic control device 57.

The ferromagnetic particle to be examined—or else a multiplicity of such particles in a bunch—can be embodied as a micro-machine, micro-robot or micro-component. A typical dimension of such a ferromagnetic particle can range from 0.02 mm to a few millimetres (in relation to the greatest diameter). The ferromagnetic particle consists, in part or in full, of ferromagnetic material, in particular iron, cobalt or nickel, or alloys of these metals.

### LIST OF REFERENCE SIGNS

50 MRI system

51 Magnet

52 Measurement volume

53 Radiofrequency excitation and read coil system

54 Gradient coil system

55 First coil subsystem

55a Main part

55b Attachment

56 Second coil subsystem

57 Control device

What is claimed is:

1. Method for determining a spatial position of at least one ferromagnetic particle in an object with an MRI system, comprising:
applying an MRI measurement sequence to a measurement volume in which the particle is situated,
recording at least two projections per spatial direction, wherein a read gradient is switched in each case for recording a one-dimensional projection in each case, and wherein a preselected parameter is modified for each of the two projections, and
determining the spatial position of the ferromagnetic particle with the aid of the two projections.

2. Method according to claim 1, wherein the preselected, modified parameter comprises a sign change in the read gradient.

3. Method according to claim 2, wherein a spin echo method is used, in which the read gradients have different signs.

4. Method according to claim 3, wherein a radio-frequency pulse for the spin echo is designed for a flip angle <90°.

5. Method according to claim 1, wherein the preselected, modified parameter comprises an echo time variation.

6. Method according to claim 1, wherein the preselected, modified parameter comprises a variation in amplitude of the read gradient while simultaneously maintaining field of view.

7. Method according to claim 1, wherein two one-dimensional projection images are recorded in each case in three different spatial directions and the spatial position of the ferromagnetic particle in the three-dimensional space is determined therefrom.

8. Method according to claim 7, wherein the three different spatial directions are mutually orthogonal spatial directions.

9. Method according to claim 1, wherein the position of the ferromagnetic particle is determined
by forming the difference of the recorded one-dimensional projection images,
by overlaying the recorded one-dimensional projection images or
through pattern recognition when comparing the recorded one-dimensional projection images to one another.

10. Method according to claim 1, wherein the ferromagnetic particle is marked with another NMR-active nucleus.

11. Method according to claim 10, wherein spin-active nuclei are used as marker material.

12. Method according to claim 11, wherein the spin-active nuclei are heteroatoms in polymers.

13. Method according to claim 12, wherein the heteroatoms in polymers are selected from F in Teflon, Cl in PVC, and N in polyurethane.

14. Method according to claim 13, wherein the heteroatoms in polymers are selected from 6Li, 10B, 14N, 15N, 17O, 19F, 23Na, 29Si, 31P, 35Cl, 113Cd, 195Pt.

15. Magnetic resonance imaging (MRI) system, comprising:
a magnet configured to produce a homogeneous NMR magnetic field B₀ in a measurement volume,
a gradient coil system configured to submit magnetic field gradients in the measurement volume, and
a radiofrequency excitation and read coil system configured to radiate radiofrequency pulses into the measurement volume and to read the measurement volume,
wherein the MRI system is configured to determine the position of a ferromagnetic particle with the method according to claim 1.
